(19) United States
(12) Patent Application Publication
(10) Pub. No.: US 2006/0027239 A1
(43) Pub. Date: Feb. 9, 2006

(54) ERECTKING

(76) Inventor: CHIH CHENG WANG, FLUSHING, NY (US)

Correspondence Address: CHIH CHENG WANG, P. O. BOX 521203, FLUSHING, NY, 11352-1203, US

(21) Appl. No.: 10/710,832

(22) Filed: Aug. 5, 2004

(51) Int. Cl. A61F 5/37

(52) U.S. Cl. 128/846

(57) ABSTRACT

The material, the width, the thickness, with the ruble's tension Plus the unique way (not a part of this patent) of applying this Erectking make this product a universal tool for the treatment or management of Erectile dysfunctions, impotence, or unsatisfied size.

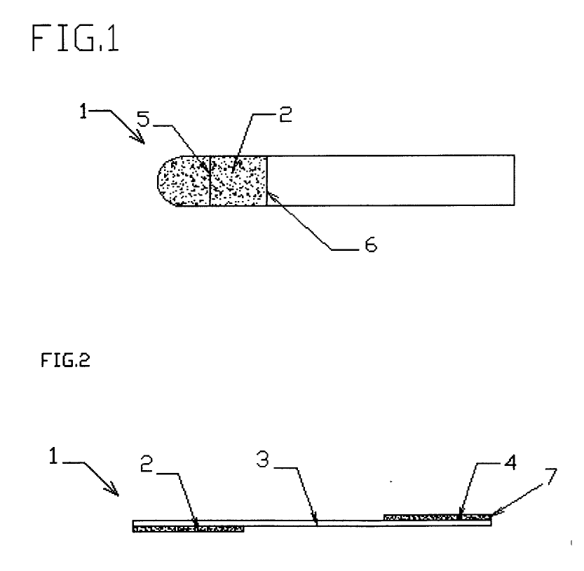

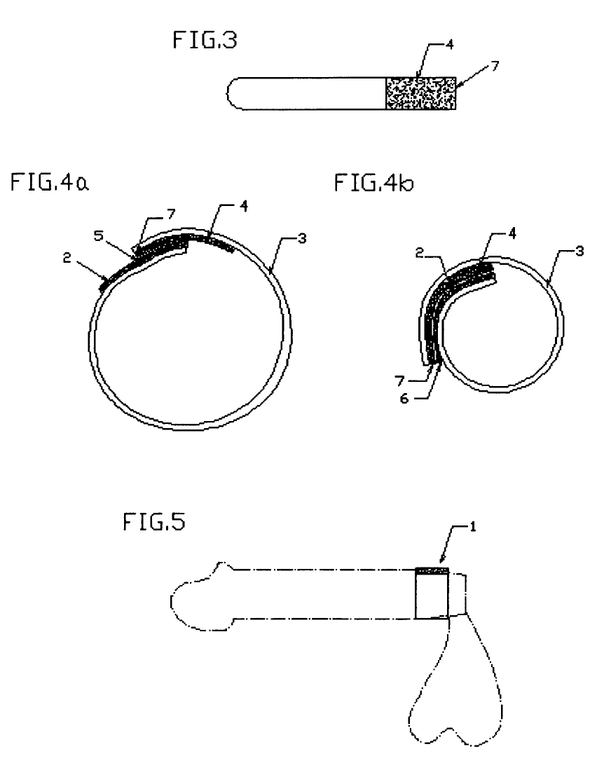

### DETAILED DESCRIPTION

Made of: The Erectking is made of ruble with male and female lock on each side and each end. The Body: made of ruble. The Base: Female lock affixed on the area. The Tip: Male lock affixed on this area.

For: the treatment or management of erectile dysfunction, impotence or unsatisfied size.

How to use it: a) Make the Erectking as a ring, by banding the Tip to touch the base part. the female and the male lock will lock and form a constructed ring. b) to release, simply pull the Tip to open the ring.

1. THE ERECTKING is my invention. I have tested it and use it since 1975.
